(19) United States
(12) Patent Application Publication
(10) Pub. No.: US 2007/0203563 A1
(43) Pub. Date: Aug. 30, 2007

(54) System for delivering a stent

(76) Inventors: Stephen Hebert, San Francisco, CA (US); Marc-Alan Levine, Pottstown, PA (US)

Correspondence Address: Neil D. Gershon, 1011 High Ridge Road, Stamford, CT, 06905, US

(21) Appl. No.: 11/703,341

(22) Filed: Feb. 7, 2007

Related U.S. Application Data
(60) Provisional application No. 60/772,660, filed on Feb. 13, 2006.

(51) Int. Cl. A61F 2/84

(52) U.S. Cl. 623/1.12

(57) ABSTRACT

A stent delivery system comprising a hypotube having a proximal end portion, a distal end portion and a first diameter, and a guidewire having a second diameter and a reduced diameter portion having a diameter smaller than the first diameter of the hypotube for receiving a stent. The guidewire is slidably positioned within the hypotube. Flexible material, e.g. collapsible tubing such as shrink tubing, extends from a portion of the hypotube to a portion of the guidewire. A stent is positioned on the reduced diameter portion of the guidewire,

This application claims priority from provisional application No. 60/772,660, filed Feb. 13, 2006, the entire contents of which are incorporated herein by reference.

### BACKGROUND

1. Technical Field

This application relates to a system for delivering a stent, and more particularly a delivery system wherein a stent is mounted on a hypotube or wire to reduce the overall profile of the system.

2. Background of Related Art

Intravascular stents are used for treatment of vascular stenosis. One type of stents are balloon expandable stents which are mounted over a balloon. Inflation of the balloon expands the stent within the vessel to dilate the stenosis. Another type of stents is self-expanding, composed of shape memory material. The self-expanding stents are compressed within a sheath and when exposed from the sheath automatically move to an expanded shape memorized position within the vessel.

These stents are delivered to the area of stenosis or an aneurysm by a catheter which is inserted over a guidewire. For balloon expandable stents, the balloon is mounted on the outside of the catheter and is expanded by injection of fluid through the catheter. Expansion of the balloon expands the overlying stent. For self-expanding stents, these stents are compressed against the outer surface of the catheter and placed inside a sheath or delivery catheter or positioned inside the delivery catheter and ejected by a catheter pusher positioned inside the delivery catheter, thereby requiring a larger diameter delivery catheter.

The applicants in an earlier application recognized that utilizing a catheter with a stent mounted thereon did not enable access to small vessels. To reduce the cross-sectional dimension of the stent delivery system, the applicants developed a system for placing a stent on the guidewire or hypotube, rather than on or in the catheter (which was inserted over a guidewire), thereby eliminating the larger dimensioned catheter. This system is described in commonly assigned U.S. Pat. No. 6,989,024, the entire contents of which are incorporated herein by reference, which discloses a stent mounted on a guidewire. The stent is mounted on a reduced diameter portion of the guidewire, resulting in an overall reduced profile. Proximal and distal radiopaque marker bands, functioning as proximal and distal stops for the stent, are also described for certain embodiments.

The apparatus and method disclosed in the '024 patent is effective in accessing smaller vessels and delivering a stent to such vessels. The present application provides improvements and variations to the stent delivery systems disclosed in the '024 patent.

### SUMMARY OF THE INVENTION

The present invention provides a stent delivery system comprising a hypotube having a proximal end portion, a distal end portion and a first diameter, and a guidewire having a second diameter and a reduced diameter portion having a diameter smaller than the first diameter of the hypotube for receiving a stent. The guidewire is slidably positioned within the hypotube. Flexible material, e.g. collapsible tubing such as shrink tubing, extends from a portion of the hypotube to a portion of the guidewire. A stent is positioned on the reduced diameter portion of the guidewire, wherein distal movement of the guidewire exposes the stent to enable it to move to its expanded position. In a preferred embodiment, the material is connected to a distal region of the guidewire.

In one embodiment, exposure of the stent from the hypotube causes the stent to expand first in an intermediate and proximal portion, followed by the distal portion being exposed from the flexible material and expanding. In another embodiment, exposure of the stent causes the stent to expand first in the intermediate and distal portion, followed by the proximal portion being exposed from the hypotube and expanding. The stent can be positioned within the hypotube proximal of the flexible material. Preferably, advancement of the guidewire and stent causes the flexible material to collapse on the guidewire. In one embodiment, the reduced diameter portion is formed by a stepped portion wherein a second portion of the guidewire is a coiled region stepping down from the hypotube and a first portion is a coiled region stepping up from the second portion.

The present invention also provides a stent delivery system comprising a hypotube having a proximal end, a distal end and a first diameter. A guidewire is slidably positioned within the hypotube and is movable from a first retracted position to a second advanced position. A stop is on the guidewire and a stent is mounted on the guidewire to limit proximal movement of the stent. A flexible material is connected to a portion of the guidewire and a portion of the hypotube, wherein movement of the guidewire to the second position detaches the flexible material from the hypotube and exposes the stent to enable it to move to its expanded position.

In one embodiment the flexible material is composed of a flexible tubing such as shrink tubing. Preferably, the stent is mounted on a reduced diameter portion of the guidewire. In one embodiment, the reduced diameter portion is formed by a first coiled region having a diameter less than a diameter of a second coiled region.

### BRIEF DESCRIPTION OF THE DRAWINGS

Preferred embodiments of the present disclosure are described herein with reference to the drawings wherein:

FIG. 1 is a perspective view of a first embodiment of the stent delivery system of the present invention;

FIG. 2 is a view similar to FIG. 1 showing retraction of the pull wire to expose the stent;

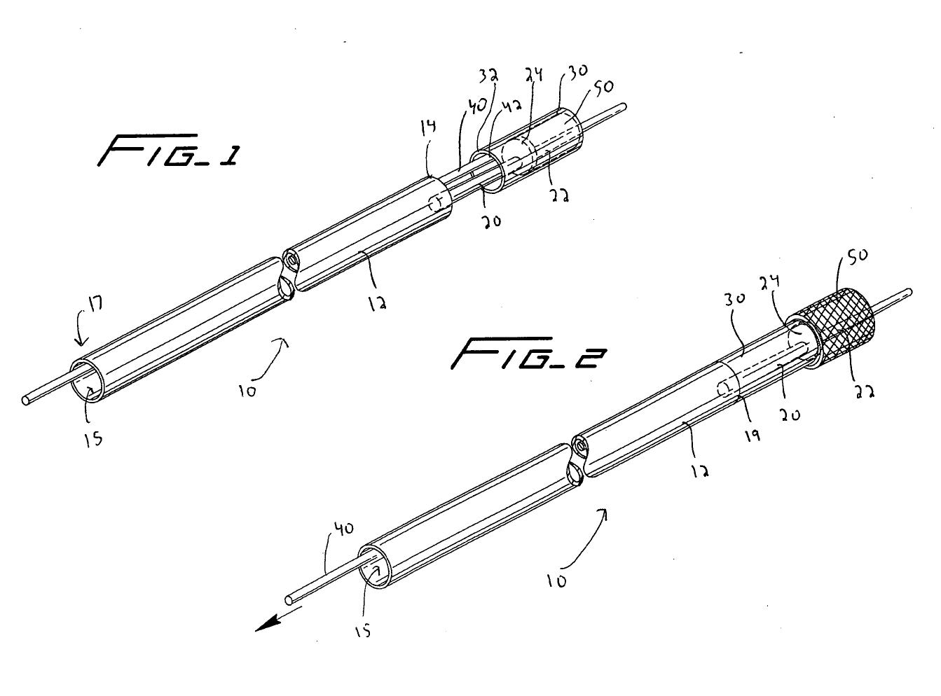

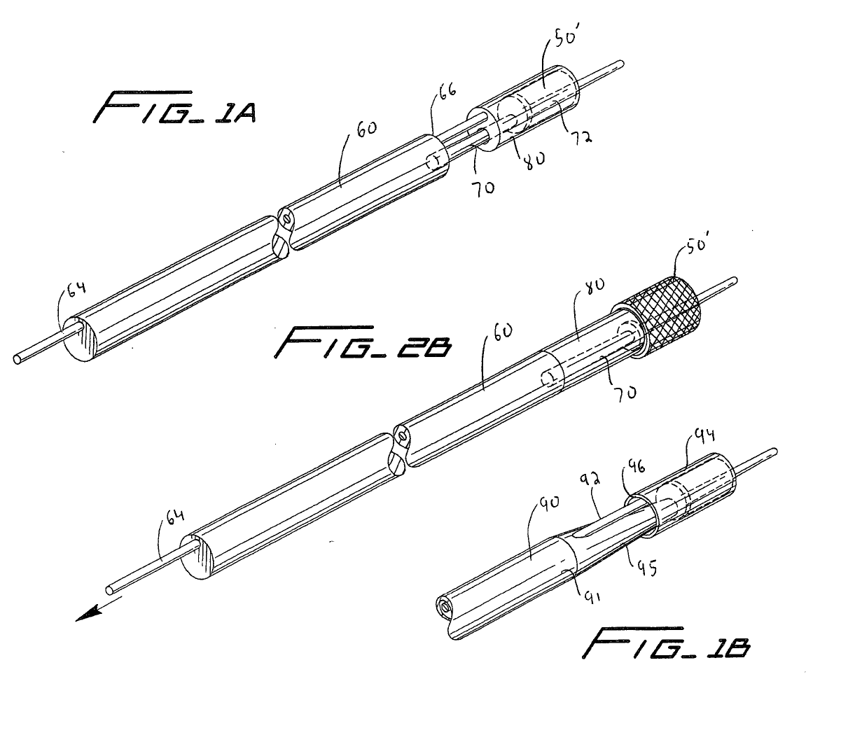

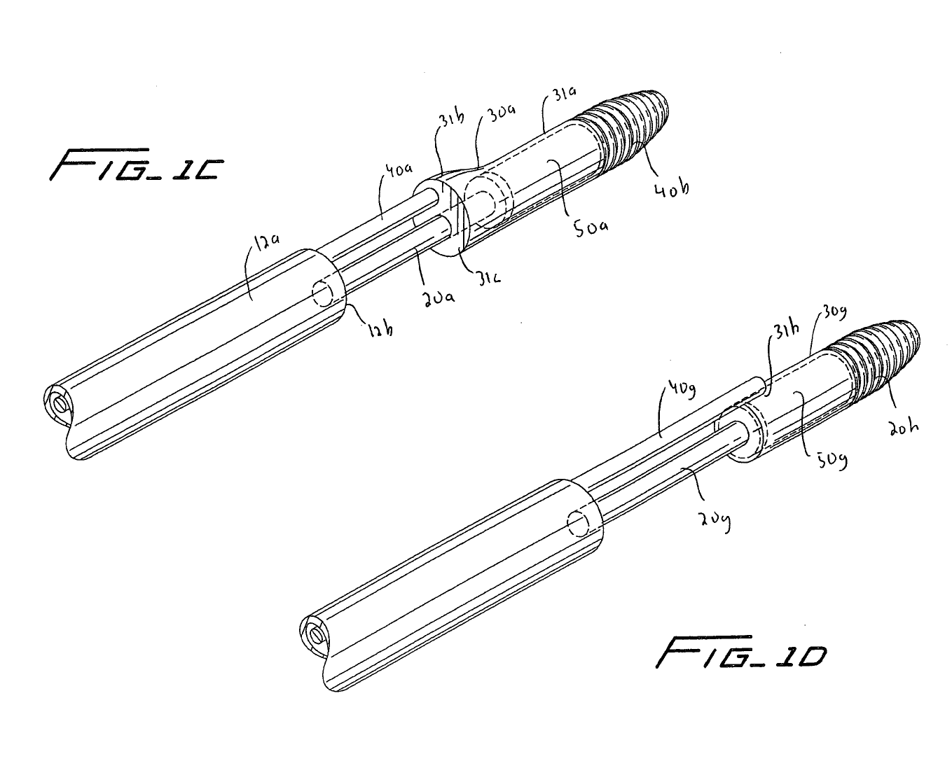

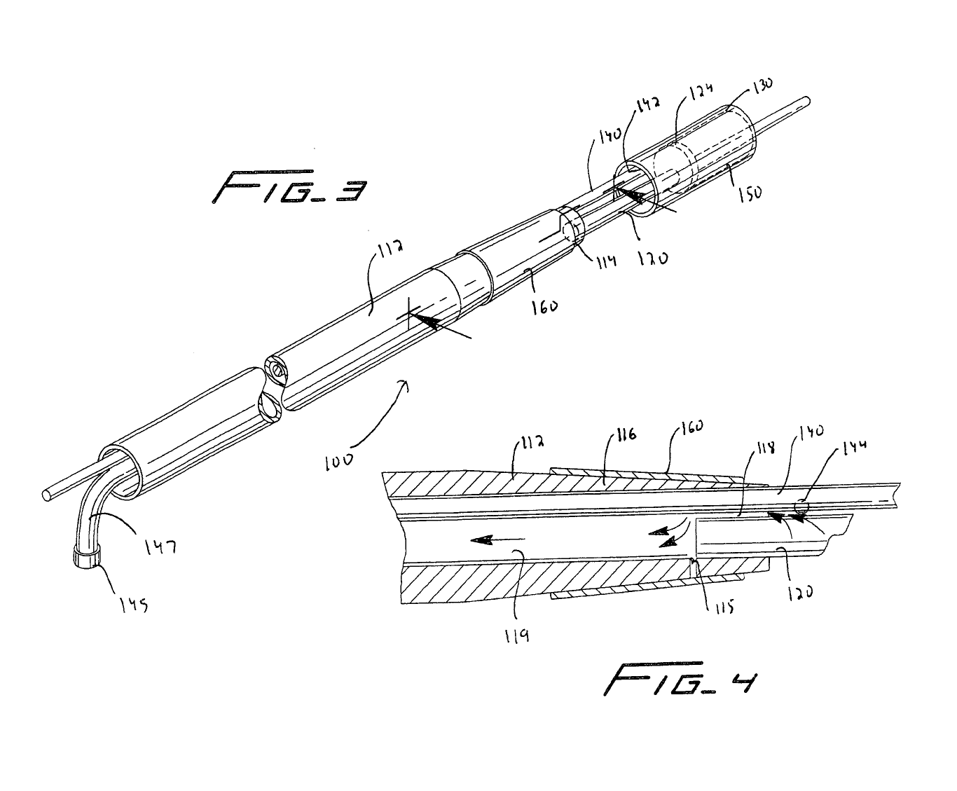

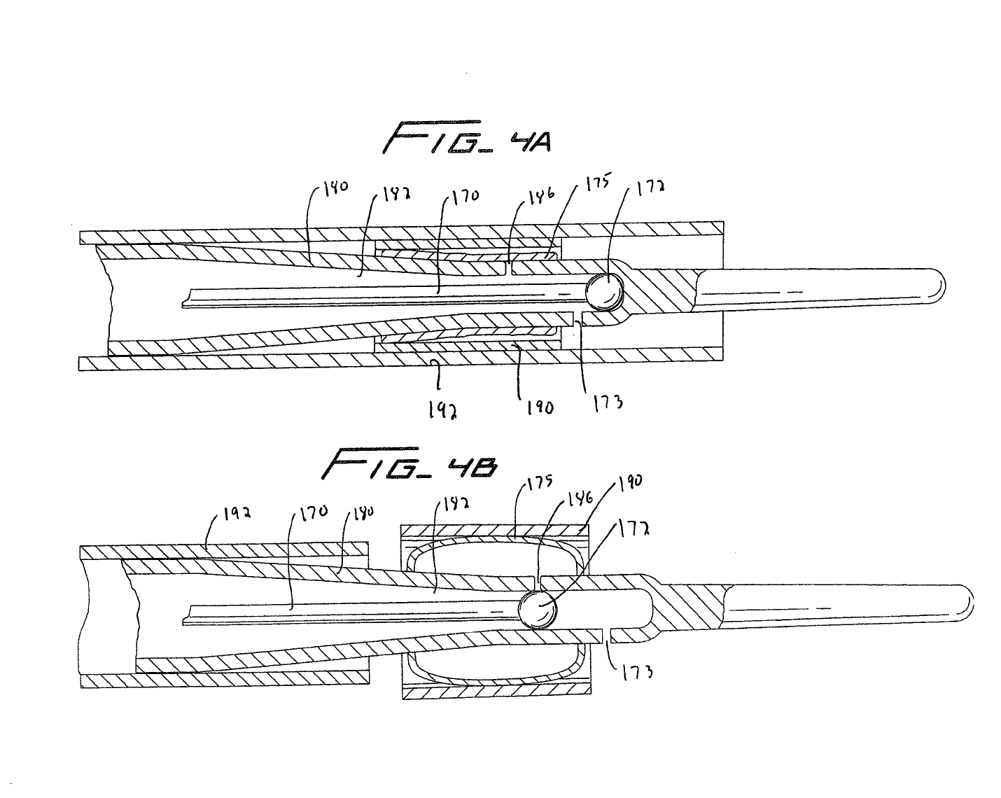

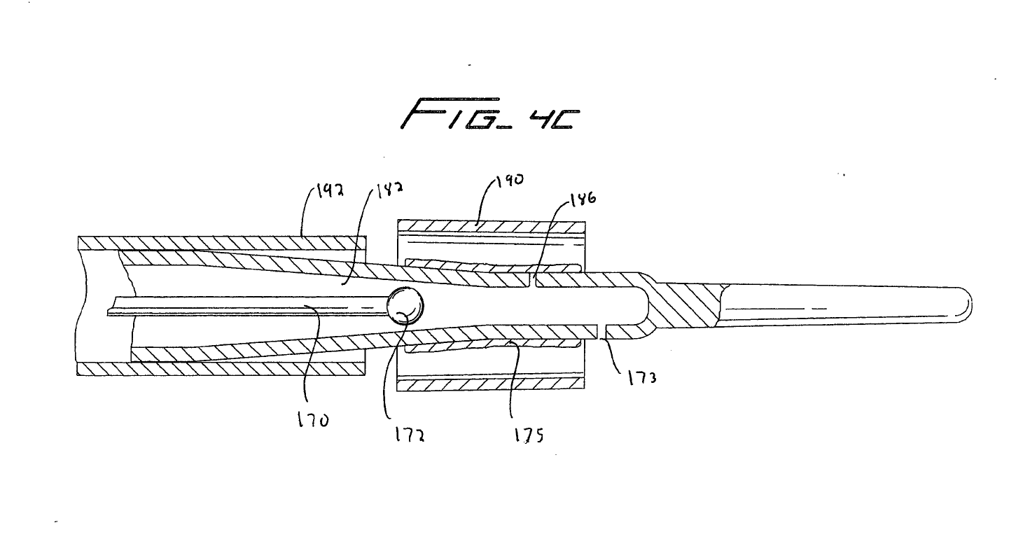

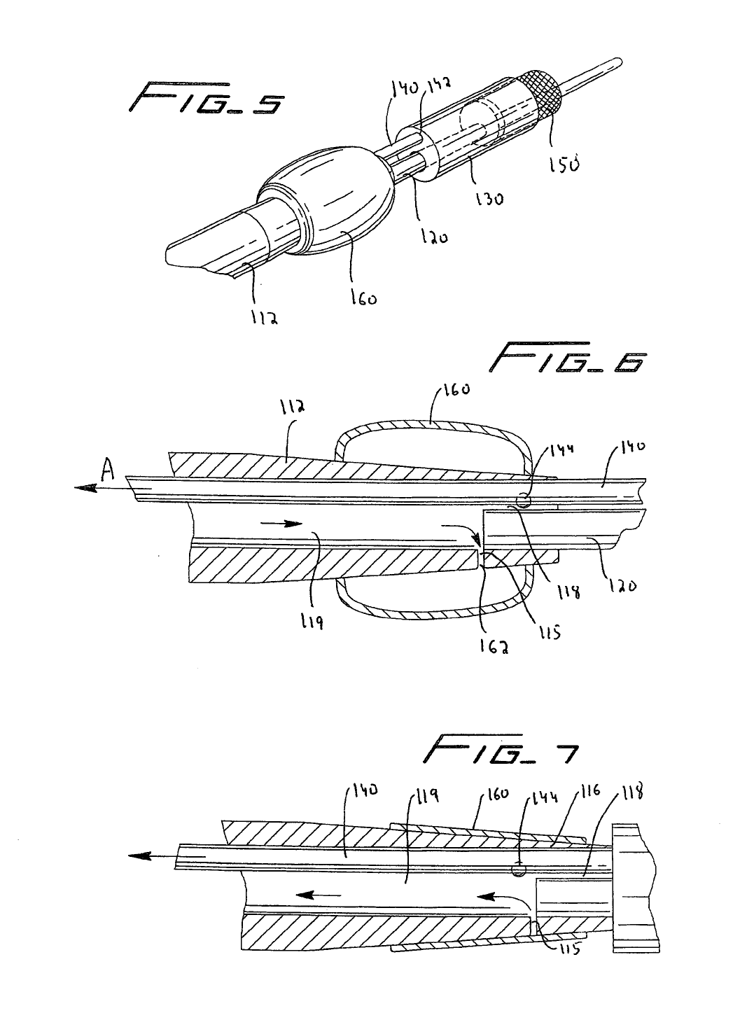

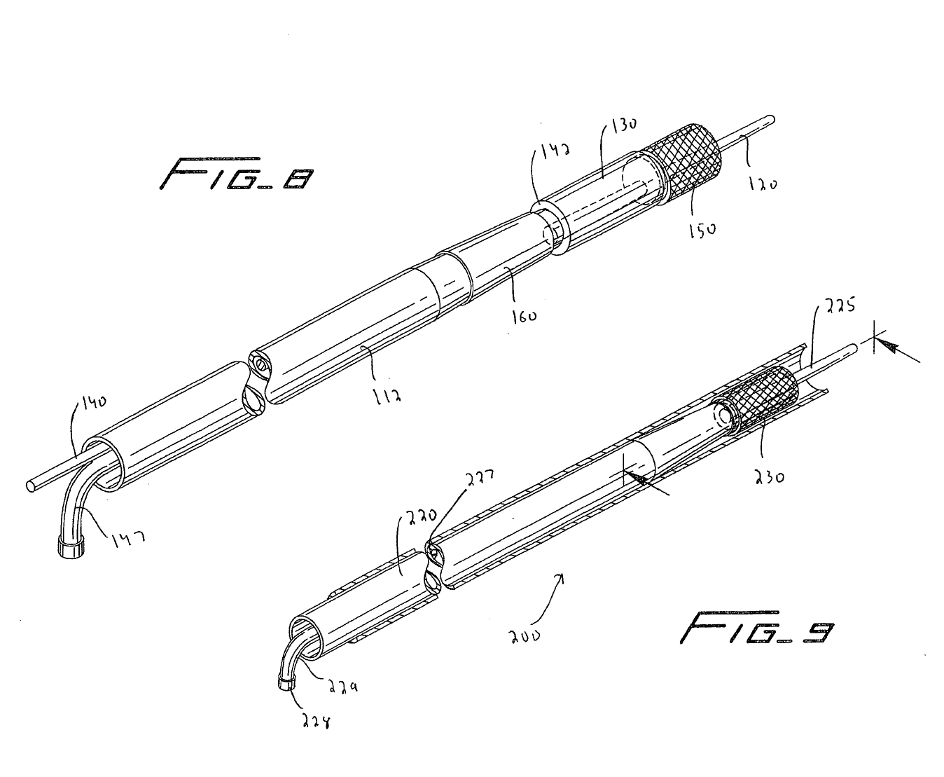

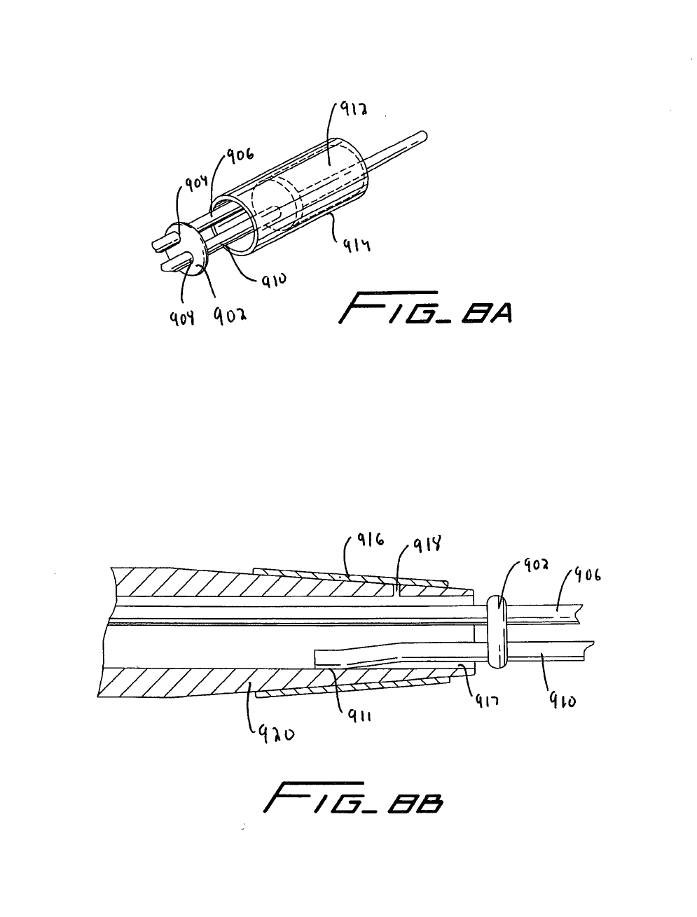

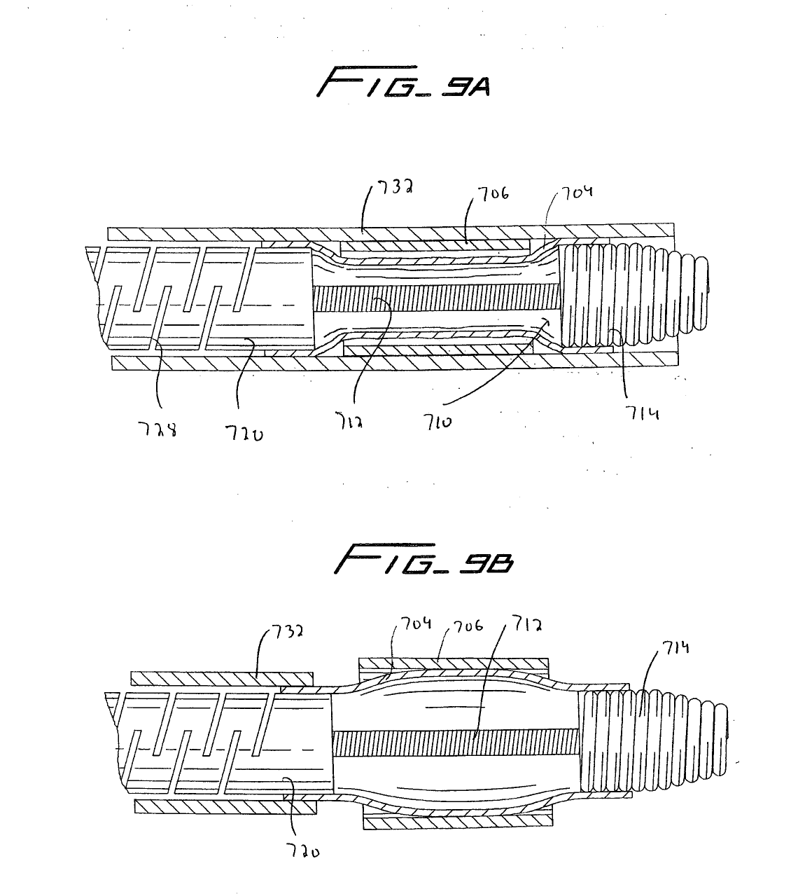

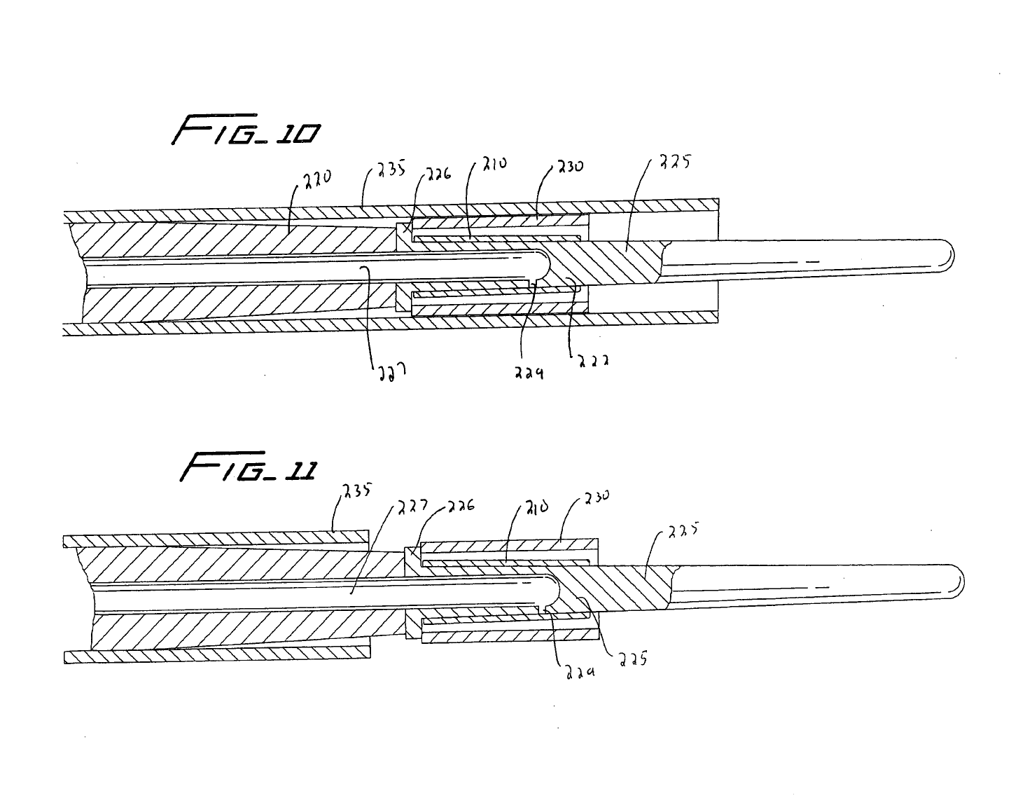

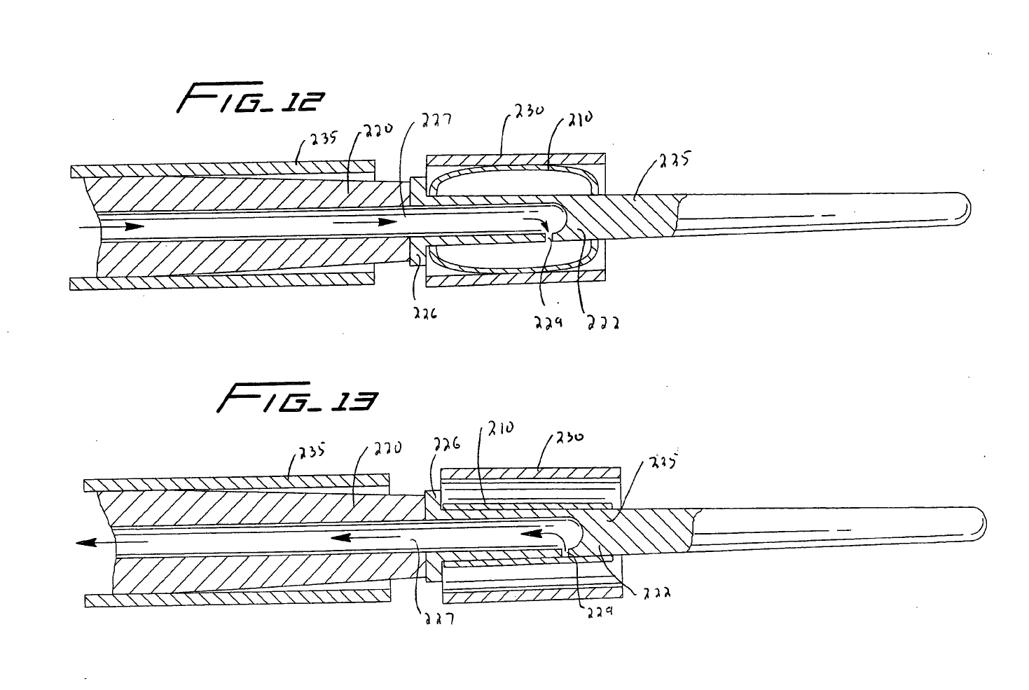

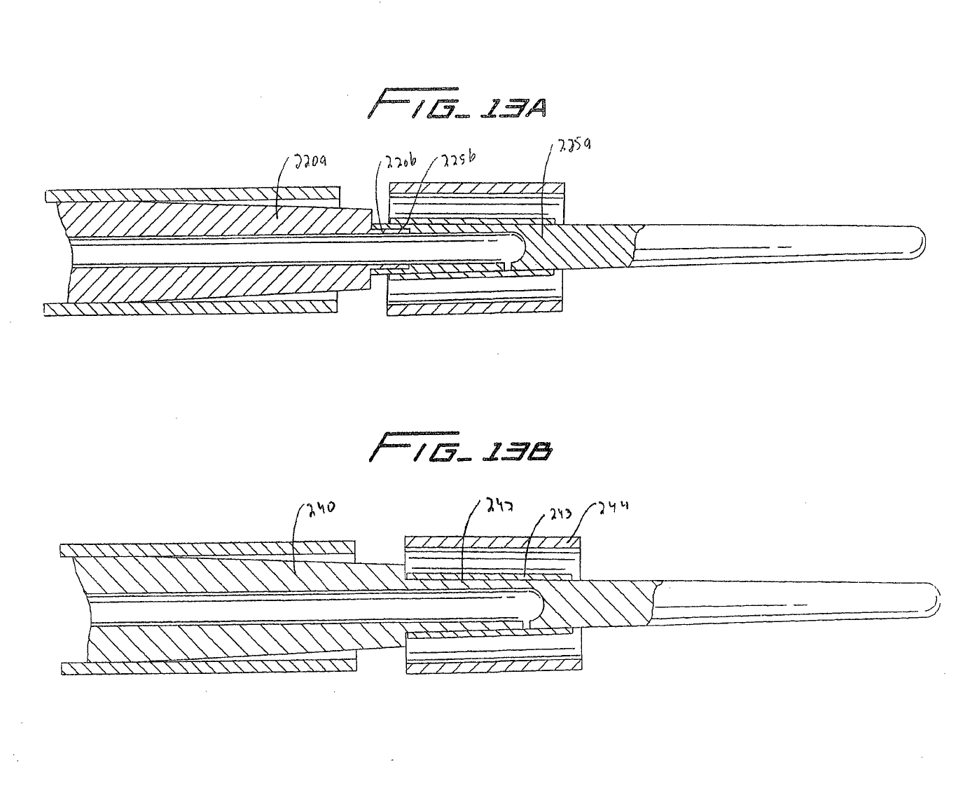

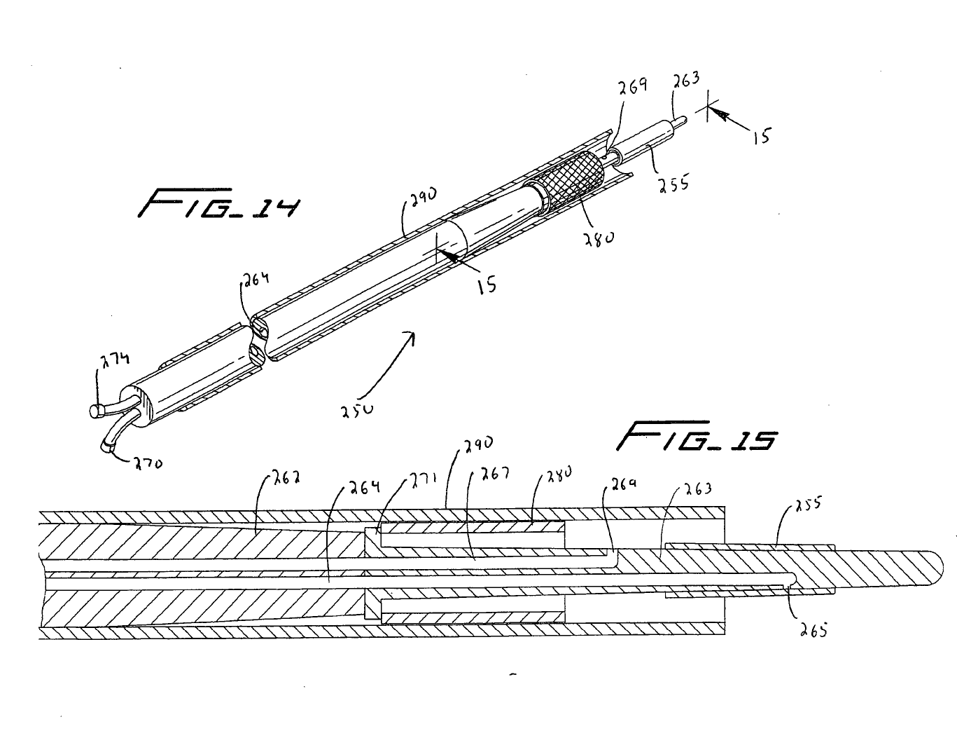

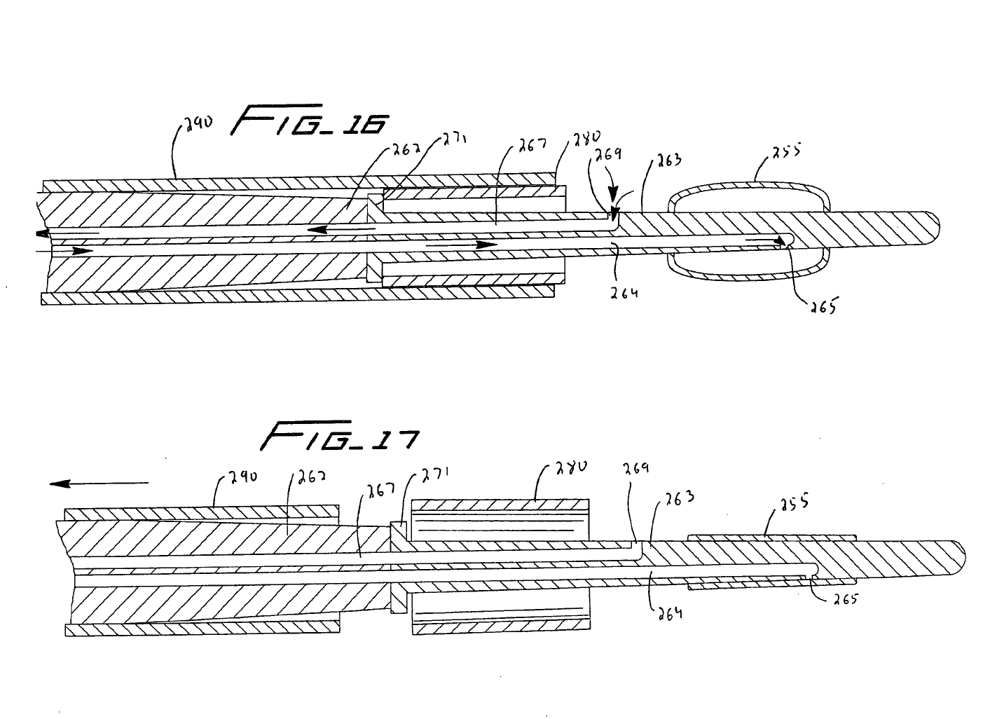

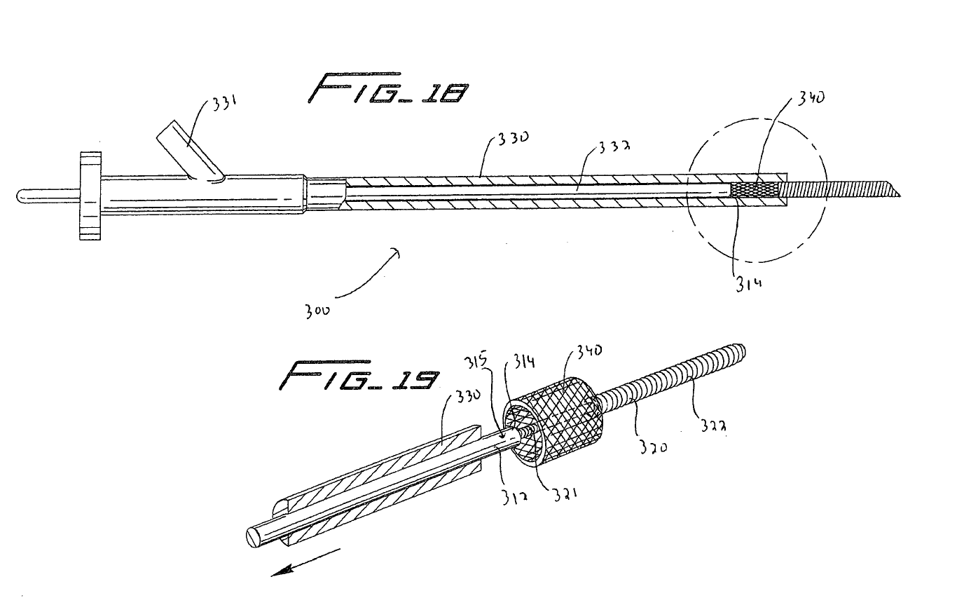

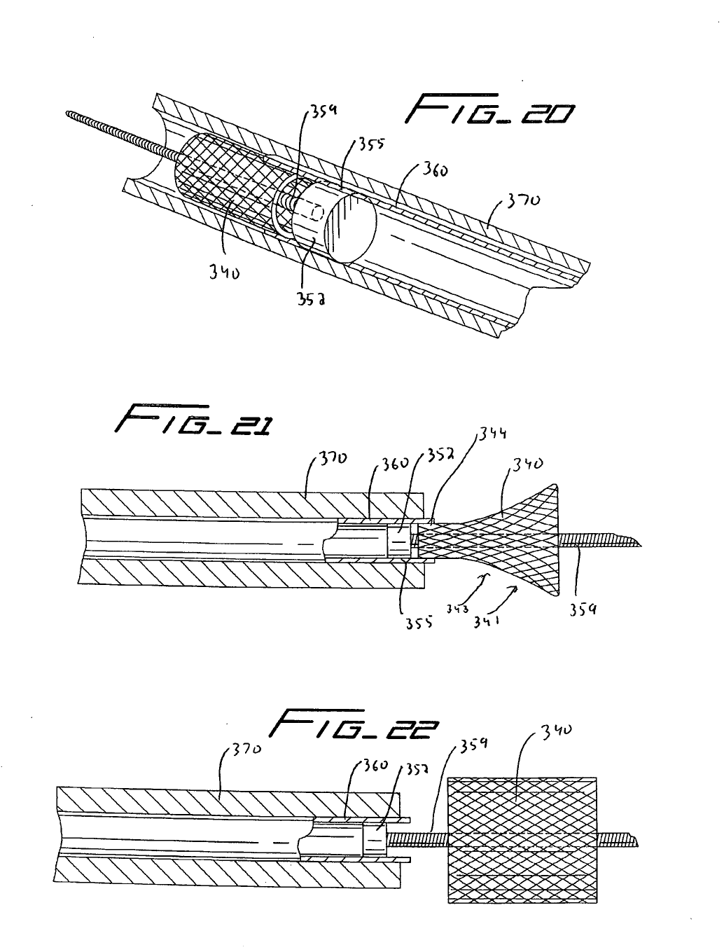

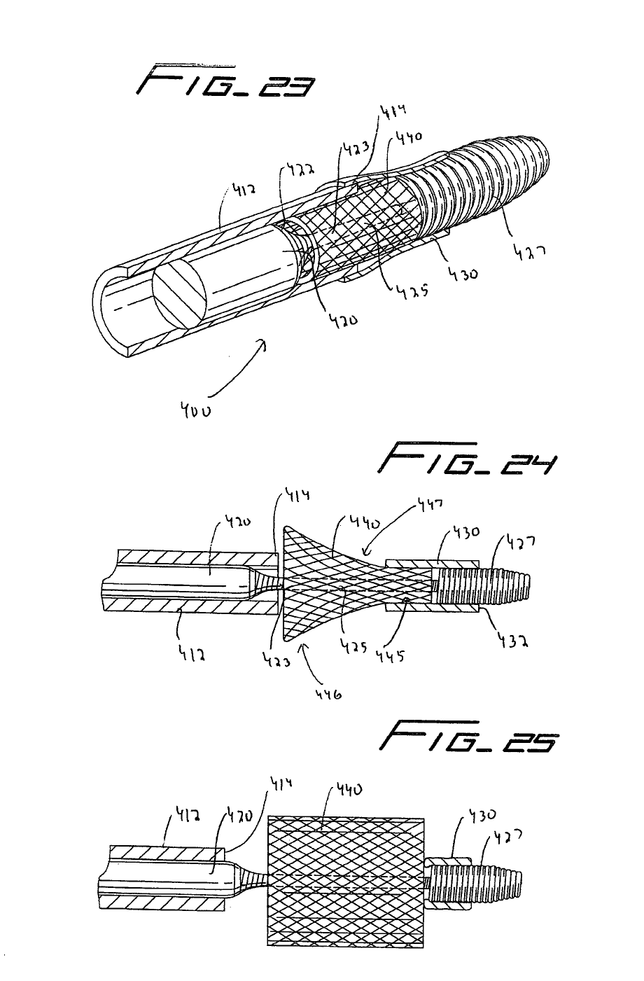

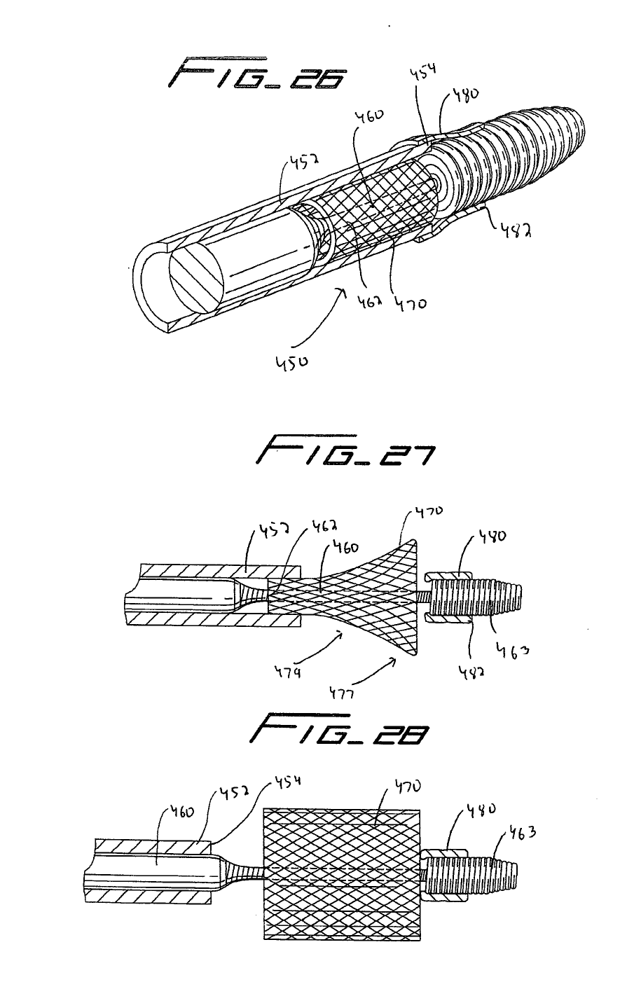

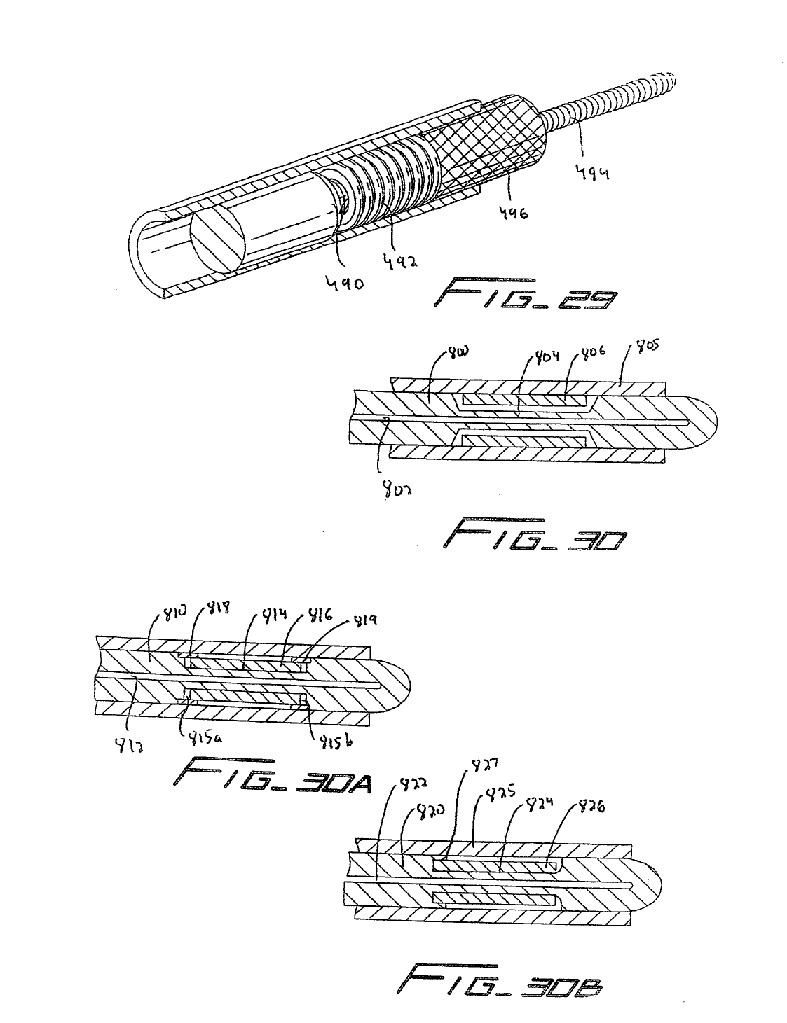

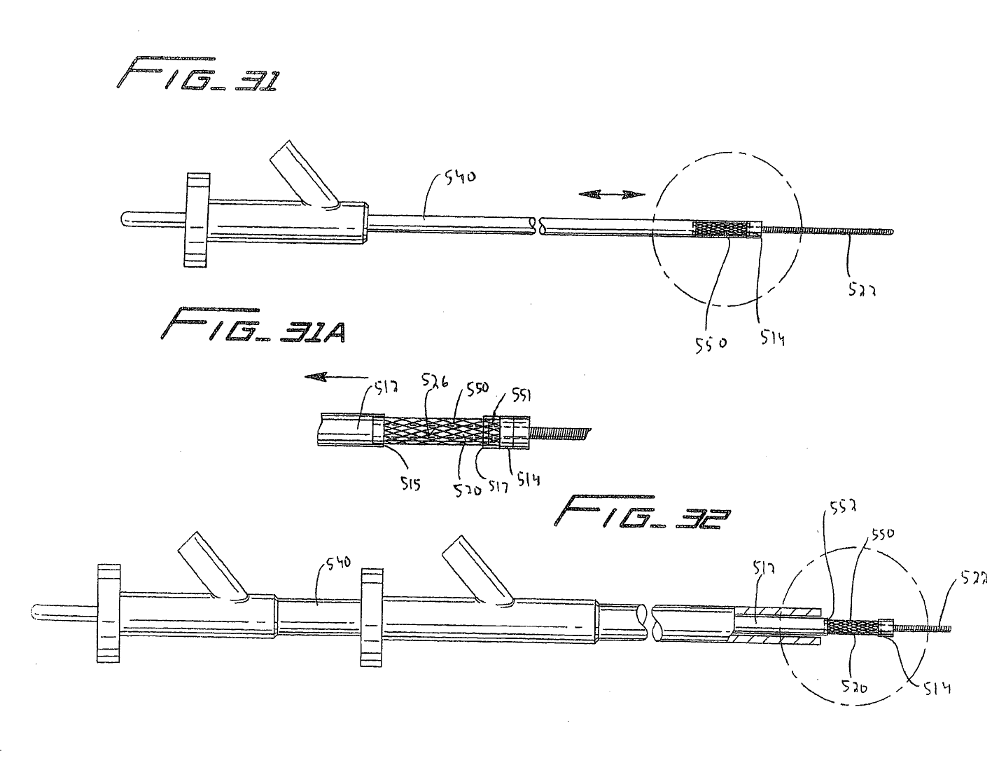

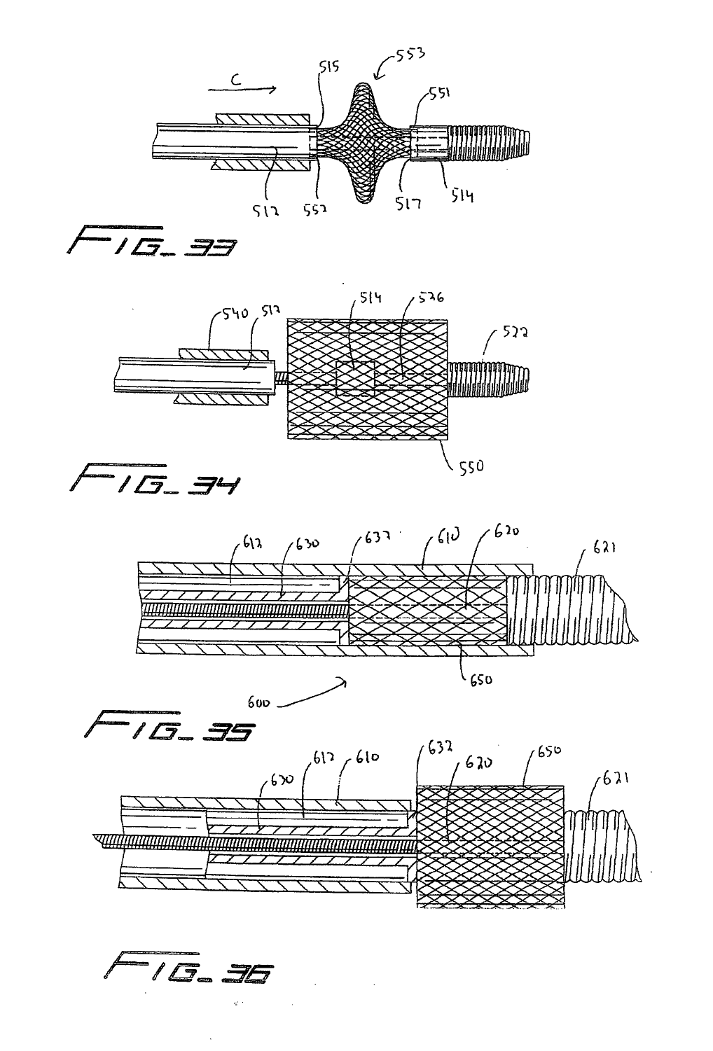

FIGS. 1A and 2B are views similar to FIGS. 1 and 2 showing an alternate embodiment utilizing a solid tube with a lumen;

FIG. 1B is a perspective view of an alternate embodiment having a tapered hypotube;

FIG. 1C is a perspective view of an alternate embodiment having a tapered sheath;

FIG. 1D is a perspective view of another alternate embodiment having the retraction wire connected to the outside of the sheath;

FIG. 3 illustrates a perspective view of another alternate embodiment of the stent delivery system of the present invention showing the balloon in the deflated condition positioned proximally of the stent and the pull wire in the distal position;

FIG. 4 is a cross-sectional view taken along lines 4-4 of FIG. 3;

FIGS. 4A, 4B and 4C are cross-sectional views of another alternate embodiment of the stent delivery system of the present invention having a ball valve at the distal end of the pull wire and shown movable from a first (distal) position to a sealing position and to a retracted unsealing position;

FIG. 5 is a broken perspective view of the distal portion of the system of FIG. 3, showing the balloon in the inflated condition and the pull wire partially retracted to expose a distal portion of the stent;

FIG. 6 is a cross-sectional view similar to FIG. 4 showing the balloon in the inflated condition, the distally directed arrows representing injection to inflate the balloon and the proximally directed arrow representing retraction of the pull wire to move the valve within the hypotube to seal off the hypotube for balloon inflation;

FIG. 7 is a cross-sectional view similar to FIG. 4 showing the balloon in the deflated condition and the pull wire in the fully retracted position to further retract the valve;

FIG. 8 is perspective view similar to FIG. 3 showing the balloon in the deflated condition and the stent exposed and in its expanded position;

FIGS. 8A and 8B are perspective and cross-sectional views, respectively of another alternate embodiment having a retractable valve;

FIG. 9 is a perspective view of another alternate embodiment of the stent delivery system of the present invention showing the stent in the compressed position and a balloon in a deflated condition underlying the stent, both the stent and balloon shown contained within the sheath (shown partially cut away);

FIG. 9A is a cross-sectional view of another alternate embodiment showing the balloon mounted on a reduced coil section of the guidewire, the stent shown in the collapsed delivery position;

FIG. 9B is a view similar to FIG. 9A showing the balloon and stent expanded;

FIG. 10 is a cross-sectional view taken along lines 10-10 of FIG. 9;

FIG. 11 is a view similar to FIG. 10 showing the sheath further retracted to expose the balloon and stent;

FIG. 12 is a view similar to FIG. 10 showing the balloon inflated to expand the stent;

FIG. 13 is a view similar to FIG. 10 showing the balloon in the deflated condition and the stent remaining in the expanded position;

FIG. 13A is a view similar to FIG. 13 except showing an alternate embodiment for attachment of the wire to the tube;

FIG. 13B is a view similar to FIG. 13A except showing another alternate embodiment;

FIG. 14 is a perspective view in partial cross-section of yet another alternate embodiment of the stent delivery system of the present invention showing a balloon in the deflated condition positioned distally of the stent, the stent shown in the compressed position within the sheath;

FIG. 15 is a cross-sectional view taken along lines 15-15 of FIG. 14;

FIG. 16 is a view similar to FIG. 15 showing the balloon in the inflated condition and the stent in the compressed position within the sheath;

FIG. 17 is a view similar to FIG. 15 showing the balloon in the deflated condition and the stent in the expanded position exposed from the sheath;

FIG. 18 illustrates another alternate embodiment of the stent delivery system of the present invention showing the stent in the compressed condition within the catheter;

FIG. 19 is a close up perspective view of the area of detail denoted in FIG. 18 showing the stent in the expanded position exposed from the catheter;

FIG. 20 is a perspective view in partial cross section of still another alternate embodiment of the stent delivery system of the present invention showing the core wire with an enlarged back end positioned within a hypotube and the stent in the compressed position;

FIG. 21 is a cross-sectional view showing partial expansion of the stent of FIG. 20;

FIG. 22 is a view similar to FIG. 21 showing full expansion of the stent;

FIG. 23 is a perspective view in partial cross section of another alternate embodiment of the stent delivery system of the present invention showing the stent in a compressed position with the distal portion covered by a shrink wrap;

FIG. 24 is a cross-sectional view of the system of FIG. 23 showing the shrink wrap advanced from the hypotube and the stent partially expanded;

FIG. 25 is a view similar to FIG. 24 showing the stent in the fully expanded position with the shrink wrap collapsed on the guidewire;

FIG. 26 is a perspective view in partial cross-section of another embodiment of the stent delivery system of the present invention showing the stent in a compressed condition;

FIG. 27 is a cross-sectional view of the system of FIG. 26 showing the stent partially expanded as it is partially advanced from the hypotube;

FIG. 28 is a view similar to FIG. 27 showing the stent in the fully expanded position;

FIG. 29 is a perspective partial cross-sectional view of another embodiment of the stent delivery system having different sized guidewire coils;

FIGS. 30-30B are perspective views of alternate embodiments of a plastic guide for mounting the stent;

FIG. 31 illustrates a side view of yet another alternate embodiment of the stent delivery system of the present invention, the stent shown in the collapsed position within the catheter;

FIG. 31A is a close up view of the area of detail denoted in FIG. 30;

FIG. 32 is a view similar to FIG. 31 showing retraction of the catheter to expose the stent;

FIG. 33 illustrates movement of the tube to expand the middle portion of the stent of FIG. 30;

FIG. 34 is a view similar to FIG. 33 showing the stent in the expanded position, released at both ends from the tubes;

FIG. 35 illustrates a cross-sectional view of still another alternate embodiment of the stent delivery system of the present invention, the stent shown in the collapsed position within the sheath; and

FIG. 36 is a view similar to FIG. 35 showing the stent in the expanded position exposed from the sheath.

### DETAILED DESCRIPTION OF PREFERRED EMBODIMENTS

Referring now in detail to the drawings wherein like reference numerals identify similar or like components throughout the several views, a first embodiment of the stent delivery system of the present invention is shown in FIGS. 1 and 2. In this embodiment, stent delivery system is represented generally by reference numeral 10 and includes a hypotube 12, a tapered core wire (guidewire) 20 extending beyond a distal end of the hypotube 12, a sheath or tube 30 covering the stent, and a control member in the form of a pull wire 40. The guide wire 20 is attached at a proximal end to the distal end region 14 of the hypotube 12 by soldering to the inside wall of the hypotube, by welding or other attachment means. As illustrated, the core wire 20 is located off center from and preferably substantially parallel to a central longitudinal axis of the hypotube 12 and has a smaller diameter than the hypotube. Pull wire 40 extends through lumen 15 in the hypotube 12 so it emerges beyond a proximal end 17 of the hypotube 12 to be manipulated by the user. The pull wire 40 is also off center from and substantially parallel to the longitudinal axis of the hypotube 12. The distal end 42 of wire 40 is attached to a proximal end region 32 of sheath 30.

Stent 50 is mounted on core wire 20 in region 22, which is preferably tapered or otherwise reduced in diameter, such as by a stepped portion (not shown in this embodiment but illustrated in other embodiments). A proximal stop 24, which can be integral with the wire 20 or a separately attached component, limits proximal movement of the stent 50 and is shown by way of example as a circular disk-like member, although other configurations are contemplated. The stop 24 can also be in the form of a radiopaque marker or coil to enhance imaging. The stop 24 has a transverse cross-section or outer diameter larger than the reduced diameter portion 22 of guidewire 20. By mounting the stent on the core wire, and on the tapered reduced diameter region 22 of the core wire, an overall reduced profile of the delivery system is achieved. The advantages of such reduced profile mounting in this embodiment as well as the other embodiments discussed below are described in detail in U.S. Pat. No. 6,989,024 and commonly assigned co-pending U.S. application Ser. No. 11/248,362, filed Oct. 11, 2005, the entire contents of which are incorporated herein by reference.

In use, after the system is inserted to the surgical site, the pull wire 40 is pulled proximally in the direction of the arrow of FIG. 2 to slide the attached sheath 30 proximally to expose the stent 50 positioned on the reduced diameter portion 22. This allows the self expanding stent, preferably composed of shape memory material such as Nitinol or elgiloy, although other shape memory materials are also contemplated, to expand from its compressed (collapsed) position within sheath 30 (as shown in FIG. 1) to its expanded configuration shown in FIG. 2. Note in the fully retracted position, the proximal end 32 of sheath 30 abuts distal edge 19 of hypotube 12, thus limiting proximal movement of sheath 30.

Note the stents of the embodiments disclosed herein can be composed of shape memory, stainless steel or other metals or metal composites and of radiopaque material.

FIGS. 1A and 2B illustrate an alternate embodiment of the stent delivery system utilizing a solid tube instead of the hollow hypotube of FIG. 1. Solid tube 60 has a lumen preferably extending substantially parallel to the longitudinal axis of the tube 60 to slidably receive pull wire 64. In other respects, the system is similar to FIG. 1, having a guidewire 70 attached to the distal end region 66 of the tube 60 (preferably extending into a small bore formed therein) and extending substantially parallel to a longitudinal axis thereof. Stent 50′ is mounted on a reduced diameter portion 72 of guidewire 70 and a solid tube 80 covering stent 50′ is controlled by pull wire 64.

The tubes and/or sheaths disclosed in the various embodiments herein could have slits. They can be composed of composite material, and can contain a Teflon liner with a soft outer jacket. The hypotube disclosed herein can be made of various materials, including for example, a composite with layered materials, a polymer fused together which can include a liner or braid.

In the alternate embodiment of FIG. 1B, instead of a gap between the tube and sheath, hypotube 90 is tapered at region 95 so that when sheath or tube 94 is retracted by pull wire 92, it slides over the tapered portion 95 of tube 90. Proximal movement of the sheath 94 is stopped when its proximal end 96 contacts matching diameter portion 91 of hypotube 90.

In the alternate embodiment of FIG. 1C, sheath 30a is tapered. The smaller diameter tapered region 31a overlies and compresses the stent 50a. The pull wire 40a is attached to the larger diameter region 31b. The wire 20a, extending from hypotube 12a, has a radiopaque region 40b at its distal tip which can be coiled as shown or made of polymeric material and/or coated with radiopaque ink. Such coil, polymeric material and coating can be utilized with the other embodiments described herein. In all other respects, the system of FIG. 1C operates similar to that of FIG. 1, e.g. pull wire 40a is retracted until proximal end 31c of sheath 30a abuts distal end 12b of hypotube 12a.

In the alternate embodiment of FIG. 1D, pull wire 40g is attached to an outer surface 31h of sheath 30g. The system of FIG. 1D in all other respects is similar to that of FIG. 1, with the pull wire 40g retracting sheath 30g to expose the stent 50g. The wire 20g has a coiled radiopaque region 20h at its distal tip. Note, as in FIG. 1C, the coil is shown with a diameter substantially equal to the diameter of the sheath, but alternatively it could be smaller. The coil could also extend back to cover the sheath.

FIG. 3 illustrates an alternate embodiment of the stent delivery system, designated generally by reference numeral 100. The system 100 includes a hypotube 112, core (guide) wire 120 extending beyond a distal end 114 of hypotube 112, a balloon 160, and a pull wire 140 for controlling sliding movement of sheath or tube 130. The hypotube 112 preferably has a tapered region 116 on which balloon 160, e.g. an angioplasty balloon, is mounted. Stent 150 is mounted on the core wire 120, preferably on a tapered or reduced diameter region. Core wire 120 extends from distal end 114 of hypotube 112 and is attached thereto. A proximal stop 124, which can be integral with the wire 120 or a separately attached component, limits proximal movement of the stent. The stop 124 can also be in the form of a radiopaque marker to enhance imaging. Injection port 145 fluidly communicates with a lumen 119 in the hypotube 112 via tube 147. The hypotube lumen 119 communicates with an opening in the balloon 160 to allow inflation of the balloon. Note in an alternate embodiment of a solid tube, a lumen would be formed to communicate with the balloon for inflation and for the pull wire.

Pull wire 140 is attached at its distal end 142 to sheath 130 by conventional attachment methods. A ball or other shaped valve 144 is mounted on or integral with pull wire 140. When the pull wire 140 is in the position of FIG. 4, the balloon 160 is deflated and the sheath 130 covers the stent 150 to hold it in the reduced diameter compressed condition. In this position, the channel 118 can form a passageway for suction in the direction shown by the arrows in FIG. 4.

When the pull wire 140 is retracted proximally in the direction of arrow A in FIG. 6 by pulling on its proximal end, ball valve 144 is moved proximally within hypotube 112 to seal off channel 118 of hypotube 112 so the inflation fluid does not exit through channel 118 but passes through the opening 115 in the hypotube 112 and through the aligned opening 162 in balloon 160 to inflate the balloon. This position of the ball valve 144 with the inflated balloon 160 is shown in FIG. 6. In this position, stent 150 remains at least partially covered by sheath 130, as shown in FIG. 5. (Note in alternate embodiments, e.g. by adjusting the length of the sheath, stent uncovering can occur simultaneously with balloon inflation.) Next, pull wire 140 is retracted further, as indicated by the arrow of FIG. 7, to pull ball valve 144 proximally within the hypotube lumen 119, proximal of channel 118, to no longer seal the channel 118 as the valve 144 is located in a larger internal diameter region of the hypotube 112. This enables deflation of the balloon 160, indicated by the proximally directed arrows within lumen 119 of FIG. 7. Sufficient proximal movement of pull wire 140 retracts the sheath 130 a sufficient distance so that the self-expanding stent 150 is uncovered, thereby allowing it to expand from its collapsed position to its expanded position shown in FIG. 8.

FIGS. 4A-4C illustrate an alternate embodiment of the stent delivery system having a ball valve 172 mounted on or integral with the distal end of concentrically positioned wire 170. Initially, the valve 172 is in the first distal position of FIG. 4A where it is forward of opening 186 and aperture 173. Aperture 173 can be used to purge the catheter in a preparatory step as well as for aspiration as it provides communication between the lumen 182 of hypotube 180 and the patient. The balloon, preferably mounted on a reduced diameter portion of the hypotube, is inflated and the valve 172 is retracted by wire 170 (FIG. 4B) to a sealing or blocking position wherein valve 172 blocks opening 186 of lumen 182 of hypotube 180 to maintain inflation of balloon 175 to expand stent 190. Note relative movement of sheath 192 of hypotube 180 exposes the balloon 175 and stent 190 for expansion as shown in FIG. 4B. When the valve is retracted further to the position of FIG. 4C by the control member in the form of wire 170, the balloon can be deflated and aperture 173 is open for communication with the patient for aspiration. Although only a single opening and single aperture is shown, multiple openings and/or apertures could be provided. A detachable proximal luer (not shown) can be provided.

FIGS. 8A and 8B illustrate another alternate embodiment having a valve 902 with an opening 904 to receive pull wire control member 906 and an opening 908 to receive guidewire 910. Stent 912 is positioned within sheath 914 which is retractable in the same manner as the sheath of FIG. 1. When the valve 902 is retracted after inflation of balloon 916 (the stent not shown in FIG. 8B), it is retracted to cover opening 918 to maintain balloon inflation. Guidewire 910 is welded or attached by other means to an inner surface of hypotube 920 at proximal end 911 and bends slightly upwardly to provide a gap 917 to accommodate retraction of valve 902 as it rides over guidewire 910.

In the alternate embodiment of FIGS. 9A and 9B, the balloon 704 and stent 706 on mounted on a reduced diameter coil portion of the guidewire. As shown, guidewire 710 has a reduced coil section 712 extending from hypotube 720. An enlarged coil section of guidewire 710 is designated by reference numeral 714. Thus, a stepped portion instead of a taper as in FIG. 10 is provided. The hypotube 720, as with the other hypotubes disclosed herein, can have cutouts 728 to increase the flexibility and steerability. As in other embodiments described herein, relative movement of the sheath 732 exposes the stent and balloon for expansion. The delivery position of the stent is shown in FIG. 9A; the expanded placement position is shown in FIG. 9B.

In the alternate embodiment of FIGS. 9-13, the hypotube 220 of delivery system 200 has a core wire 225 attached to and extending distally therefrom. An enlarged region 226 can form the attachment area as well as provide a proximal stop for the stent (which could also be radiopaque for imaging) similar to stop 24 described above. Balloon 210 is mounted on a tapered or otherwise reduced diameter portion 222 of core 225 and underlies stent 230. The tapered portion 222 reduces the overall profile of the system 200. In an alternate embodiment, instead of an attached core wire, the hypotube itself would have a reduced diameter portion with a balloon mounted thereon and a stent overlying the balloon, as shown in FIG. 13B. The stent 230 can be a self expanding stent, such as of shape memory material, with the balloon inflated to further expand the stent once self-expanded. The stent can alternatively be a balloon expandable stent, relying on the balloon inflation for expansion. As with the other embodiments disclosed herein, the stent 230 is preferably mounted on the reduced diameter region 222 which could include a taper, a stepped down region or other structure. The injection port and tube for the inflation fluid for the balloon 210 is designated by reference numerals 228, 229, respectively, and communicates with lumen 227 of hypotube 220 which has an opening 229 aligned with an opening in the balloon 210. The hypotube 220 can be hollow or solid with a lumen formed therein as described above with other embodiments. A sheath or catheter which maintains the stent in the compressed position by preventing expansion of the balloon and/or stent is designated by reference numeral 235.

In use, retraction of the sheath or catheter 235 (or advancement of the hypotube 220 or opposite movement of both) enables balloon expansion and stent expansion by exposure of the stent 230 and balloon 210, as shown in FIG. 12. After expansion of the stent 230, the balloon is deflated (see arrows of FIG. 13) and the delivery system withdrawn, leaving the stent in the vessel.

FIG. 13A illustrates an alternate way to attach the guidewire to the hypotube. In FIG. 13A, hypotube 220a has an overcut 220b over which the undercut 225b of core wire 225a overlies and is attached thereto. In the embodiment of FIG. 13B, hypotube 240 has a reduced diameter portion in the form of a stepped down portion 242 with a balloon 243 mounted thereon and a stent 244 overlying the balloon 243. Hypotube also tapers as shown.

In the alternate embodiment of FIGS. 14-17, delivery system 250 has a hypotube 262, shown solid with lumens 264 and 267 formed therein, but alternatively could be hollow as in FIG. 1 with separate tubes positioned therein to form the lumens/passageways. As shown in FIG. 15, a tapered core wire 263 extends from a distal end of hypotube 262 and has an enlarged region 271, attached to hypotube 262, similar to region 226 of the FIG. 9 embodiment to form a stop. Instead of an attached core, alternatively, the hypotube itself could be tapered. Lumen 264 communicates with opening 265 for fluid injection to inflate balloon 255 and suction lumen 267 communicates with opening 269 for aspirating clot (see arrows of FIG. 16). Injection port 270 communicates with the inflation lumen 264 and suction port 274 communicates with suction lumen 267. Balloon 255 is shown positioned distally of the stent 280 and is inflated to block the vessel lumen to enable aspiration through hole 269 in hypotube 262. Proximal stop 271 is provided on core 263 and functions as described above with respect to stop 24. A sheath or catheter is designated by reference numeral 290. Stent 280 is mounted on a tapered or otherwise reduced diameter portion of the core wire 263 as is the balloon 255. A coiled radiopaque wire (not shown) for imaging can be provided on the hypotube distal tip or core wire in this embodiment as well as the other embodiments described herein.

Relative movement of sheath 290 and hypotube 262, e.g. retraction of sheath 290, advancement of hypotube 262 or movement of both in opposite directions; exposes stent 280 for self-expansion. FIG. 16 illustrates the balloon 255 expanded with the stent 280 remaining in the compressed or collapsed position within the sheath 290. FIG. 17 illustrates the stent 280 exposed from the sheath 290 and in its expanded configuration, with the balloon 255 deflated so it can be withdrawn through the stent so the system can be removed from the body.

FIGS. 18 and 19 illustrate another alternate delivery system designated generally by reference numeral 300. System 300 includes a hypotube 312, a wire (guidewire) 320, and a sheath 330. Preferably the hypotube 312 is closed, e.g. by soldering, at both ends to form a closed tube. Wire 320 is attached to the distal end 314 of hypotube such as by soldering or other means to extend distally therefrom and preferably has a region of smaller diameter than the hypotube 312. By way of example, the hypotube could have a diameter of about 0.008 inches to about 0.043 inches, and preferably about 0.016 inches, and the wire could have a diameter of about 0.003 inches to about 0.040 inches, and preferably about 0.016 inches, with the stepped down or reduced diameter area preferably of about 0.0095 inches. The distal edge 314 of the hypotube 312 can act as a proximal stop to limit proximal movement of the stent 340 which is mounted on the reduced diameter coil section 321 of wire 320 inside sheath 330. The larger distal coil section 322, in a preferred embodiment, has a diameter substantially equal to the diameter of the hypotube at distal section 315, although alternately it could be of larger or smaller diameter than the hypotube. Mounting of the stent 340 on the smaller diameter wire, in the stepped down region formed on the coiled section 321 between larger diameter hypotube 312 and larger diameter coil section 322, reduces the overall profile of the system as described above. Sheath or catheter 330 is slidable relative to the hypotube 312 and wire 320 to expose the stent 340 to enable it to self expand from its compressed condition of FIG. 18 to its expanded position of FIG. 19. An injection port 331 can optionally be provided.

FIGS. 20-22 illustrate an alternative way of mounting a smaller diameter core wire to a hypotube. The core wire 359, shown in this embodiment as coiled, has an enlarged back end 352 which is soldered to the hypotube 360 at region 355. This enlarged region 352 also functions as a stop to limit proximal movement of the stent. The hypotube 360 is inserted through a catheter or sheath 370 which maintains the stent 340 in a compressed position. When sheath 370 is retracted and/or hypotube 360 advanced, the distal end 341 of the stent 340 is exposed causing it to self-expand as shown in FIG. 21. After the distal and intermediate portions 341, 343 of stent 340 expand, the proximal end 344 is pulled by the expansion force of the stent from the confines of the hypotube 360 so the stent 340 moves to the expanded position shown in FIG. 22. The hypotube can be pulled back to further aid expansion.

FIGS. 23-25 illustrate another alternate delivery system designated generally by reference numeral 400. Core wire or guidewire 420 has a tapered or otherwise reduced region, beginning at transition region 422 and extending into first coil section 423, to form a reduced diameter region 425 for mounting stent 440 in a low profile manner. The larger diameter distal coil region of wire 420 is designated by reference numeral 427. Thus, the reduced diameter portion of FIGS. 23-25 is formed by the core stepping down to a reduced diameter coil section 423 and stepping up to larger diameter coil region 427. Guidewire 420 is slidably positioned within hypotube 412. Flexible material, illustratively shown as shrink tubing 430, is attached at its distal end 432 to guidewire 420 (at radiopaque coil region) and positioned over distal end 414 of hypotube 412 to provide a smoother transition. The tubing 430, which extends from the guidewire to the hypotube, could optionally be attached to the outer distal region of the hypotube 412.

When guidewire 420 is slid forward to carry stent 440 past the distal end 414 of hypotube 412, shrink tubing 430 will disengage from hypotube 412 as shown in FIG. 24 by the force of guidewire 420. The stent 440, being exposed from the hypotube 412, can expand from its compressed position. The stent 440 will expand first in its middle region 447 and then the proximal region 446 (since its distal region 445 initially remains within shrink tubing 430), followed by expansion of the distal region 445 due to the expansion forces of the other portions of the stent 440. The wire can optionally be pushed forward to further release it from the tubing. FIG. 24 shows the proximal and intermediate portions 446, 447, respectively, of the stent 440 expanded and the distal region 445 initially held with the tubing 430; FIG. 25 shows full expansion of the stent 440 as the expansion forces cause the distal region 445 to free itself from the tubing 430. Shrink tubing has collapsed on guidewire 420. Note the radiopaque coils are shown at a distal region of core wire 420. In alternate embodiments, the coil(s) can extend along a larger region of wire 420 and as long as the whole length. Also different sized coils can be provided. An example of different sized coil regions is shown in FIG. 29 where guidewire 490 has large coiled region 492 and smaller coiled region 494. Stent 497 is positioned distal of region 492 and on region 494. Such coil of different lengths and different sizes can be used in each of the embodiments described herein.

In the alternate embodiment of FIGS. 26-28, delivery system 450 has a hypotube 452, a tapered core or guide wire 460, and flexible material, shown as shrink tubing 480, attached at a distal end 482 to the wire 460 and positioned over the distal end 454 of hypotube 452 to provide a smoother transition. The tubing 480, which extends from the guidewire to the hypotube, could optionally be attached to the outer distal region of the hypotube 452. Stent 470 is positioned on a tapered or otherwise reduced diameter coiled portion 462 of wire 460 (at radiopaque coil region) proximal of shrink tubing 480. As shown, coiled portion 462 steps up to larger diameter coiled portion 463. As the wire 460 is moved forward to expose the stent from hypotube 452, shrink tubing 480 collapses on the distal region 461 (which includes enlarged coil section 463) of wire 460 and the distal and intermediate regions 477, 479, respectively, of the stent 470 expand as shown in FIG. 27. The wire 460 continues to be pushed forward until stent 470 extends past the distal end 454 of hypotube 452 to expose the stent to enable it to self expand to the expanded configuration (see FIG. 28). Note the radiopaque coil is shown at a distal region of core wire 420. As noted above, in alternate embodiments, it can extend along a larger region of wire 420, including the whole length. Also different sized coils can be provided such as large coil region and smaller coil region.

Instead of a shrink tubing in FIGS. 23-28, an elastomeric tube could be utilized, as well as other materials which can collapse, for example, Nitinol, silicone, composite silicone, and a coil tube.

In the alternate embodiment of FIG. 29, a large coil region 492 of core wire 490 is positioned behind the stent 494. The stent 494 is positioned on a reduced diameter coiled section 496, preferably tapered in a distal direction although alternatively could be of a substantially uniform reduced diameter.

FIG. 30-30B illustrate alternate embodiments of a plastic guidewire for mounting the stents. In FIG. 30, guide 800 has a core wire 802 embedded therein. Stent 806 is mounted on the reduced diameter cut out or stepped region 804. In FIG. 30A, plastic guide 810 has a core wire 812 and a cut out or stepped region 814 for mounting the stent 816. Radiopaque distal and proximal marker bands 819, 818 are provided. A gap 815a, 815b can optionally be provided between the ends of the stent 806 and guide 810 to allow slight axial movement of the stent 806 to aid release of the stent. In FIG. 30B, the plastic guide 820 has a core wire 822 and reduced diameter region in the form of a stepped or cut out region 824. A slot 827 is molded into the plastic to receive a proximal portion 827 of stent 826 underneath to retain the proximal portion. The stents in these embodiments are shown in the collapsed position within respective sheaths 805, 815 and 825.

In the embodiment of FIGS. 31-34, the delivery system includes a hypotube 512 and a smaller diameter guidewire 520 having a radiopaque coil 522 for imaging at its distal end. A hypotube 514 or alternatively a marker band or other radiopaque member is soldered or otherwise attached to the guidewire 520 adjacent or over the radiopaque coil 522. A stent 550 is positioned coaxially on region 526 of guidewire 520 (which optionally could be of further reduced diameter). In the delivery position of the system, the distal end 551 of the stent 550 is pressed within the open proximal end 517 of hypotube 514 and the proximal end 552 of the stent 550 is pressed within the open distal end 515 of hypotube 512. Hypotube 512 is then pulled proximally in the direction of arrow 13 to stretch and collapse the stent 550 to the delivery position of FIG. 31.

The hypotube 512 and guidewire 520 are then inserted through catheter or sheath 540. To deploy the stent 550, hypotube 512 is moved distally in the direction of arrow C in FIG. 33. This expands the middle region 553 of stent 550 which then releases the distal and proximal ends 551, 552 from tubes 514, 512, by the expansion force of the middle region 553 to enable expansion of the stent 550 to the position shown in FIG. 34. The hypotube or wire can be moved to aid expansion. As with the other embodiments described herein, after stent expansion, the delivery system is withdrawn leaving the expanded stent in the vessel.

In the embodiment of FIGS. 35 and 36, the stent delivery system is designated by reference numeral 600 and includes a microcatheter, hypotube or sheath 610, a guidewire 620 and a plunger 630. Stent 650 is mounted on the guidewire 620, preferably on a reduced diameter coiled region, distal of the distal edge 632 of plunger 630 and proximal of enlarged coiled region 621. The distal edge 632 also functions as a stop to limit proximal movement of the stent 650. The stent is maintained in a compressed configuration by the microcatheter 610 when the guidewire 630 is inserted therein. Thus the stent 650 is captured between coiled region 621 and plunger 630 so the stent can be retracted within the sheath. When plunger 630 is advanced distally, the stent 650 is moved distally along the guidewire 620 past the distal edge of the microcatheter 610 to enable expansion. The guidewire 620 can be inserted into lumen 612 of an already placed microcatheter or alternatively can be inserted into the microcatheter or hypotube before its placement and then the assembly inserted to the surgical site.

In the foregoing embodiments of changing diameter hypotubes, the diameter of the hypotube may preferably range from about 0.20 inches at its largest portion to about 0.010 inches at its smallest portion, and more preferably about 0.019 inches and about 0.015 inches, respectively.

In the foregoing embodiments, the sheath which constrains the stent (and balloon) can be inserted with the stent mounted hypotube/guidewire as a single system. Alternatively, the sheath can be placed in the body, and the stent mounted hypotube/guidewire delivered through the already placed sheath.

While the above description contains many specifics, those specifics should not be construed as limitations on the scope of the disclosure, but merely as exemplifications of preferred embodiments thereof. For example, the hypotubes of the foregoing embodiments could include slots for flexibility. A metal or reinforced plastic tube could be utilized. Also in the foregoing embodiments, a distal stop, either integral or attached, and made of a radiopaque material for imaging, could be provided. Further, to provide a reduced diameter mounting region, as an alternative to a taper, a stepped region or cut out region could be provided. The tip of the wire could be shapeable. Additionally, to expose the stent, the stent mounted hypotube/guidewire could be advanced from the sheath, the sheath could be retracted, or both could be moved in opposite directions. Those skilled in the art will envision many other possible variations that are within the scope and spirit of the disclosure.

What is claimed is:

1. A stent delivery system comprising:
a hypotube having a proximal end, a distal end and a first diameter;
a guidewire having a second diameter and a reduced diameter portion for receiving a stent, the reduced diameter portion having a diameter smaller than the first diameter of the hypotube and the second diameter of the guidewire, the guidewire slidably positioned within the hypotube;
a flexible material extending from a portion of the hypotube to a portion of the guidewire; and
a stent positioned on the reduced diameter portion of the guidewire, wherein distal movement of the guidewire exposes the stent to enable it to move to its expanded position.

2. The stent delivery system of claim 1, wherein the flexible material is connected to a distal region of the guidewire.

3. The stent delivery system as recited in claim 2, wherein the stent has a proximal portion, a distal portion and an intermediate portion, wherein exposing the stent causes the stent to expand first in the intermediate and proximal portion, followed by the distal portion being exposed from the flexible material and expanding.

4. The stent delivery system as recited in claim 1, wherein the stent is positioned within the hypotube proximal of the flexible material.

5. The stent delivery system of claim 1, wherein a portion of the stent directly underlies a portion of the flexible material.

6. The stent delivery system as recited in claim 2, wherein the stent has a proximal portion, a distal portion and an intermediate portion, wherein exposing the stent causes the stent to expand first in the intermediate and distal portion, followed by the proximal portion being exposed from the hypotube and expanding.

7. The stent delivery system as recited in claim 2, wherein advancement of the guidewire and stent causes the flexible material to collapse on the guidewire.

8. The stent delivery system as recited in claim 1, wherein the flexible material is a tubing.

9. The stent delivery system as recited in claim 1, wherein the tubing is a shrink tubing.

10. The stent delivery system of claim 1, wherein the reduced diameter portion of the guidewire is formed by a stepped portion formed by first and second coiled regions of different diameters.

11. The stent delivery system of claim 1, wherein the reduced diameter portion is formed by a stepped portion wherein a second portion of the guidewire is a coiled region stepping down from the hypotube and a first portion is a coiled region stepping up from the second portion.

12. A stent delivery system comprising:
a hypotube having a proximal end, a distal end and a first diameter;
a guidewire slidably positioned within the hypotube and movable from a first retracted position to a second advanced position;
a stop on the guidewire;
a stent mounted on the guidewire, the stop limiting proximal movement of the stent; and
a flexible material connected to a portion of the guidewire and a portion of the hypotube, wherein movement of the guidewire to the second position detaches the flexible material from the hypotube and exposes the stent to enable it to move to its expanded position.

13. The stent delivery system of claim 12, wherein the flexible material is composed of a flexible tubing.

14. The stent delivery system of claim 13, wherein the flexible tubing is shrink tubing.

15. The stent delivery system of claim 12, wherein the stent is mounted on a reduced diameter portion of the guidewire.

16. The stent delivery system of claim 15, wherein the reduced diameter portion is formed by a first coiled region having a diameter less than a diameter of a second coiled region.
